(19) United States
(12) Patent Application Publication
(10) Pub. No.: US 2023/0410980 A1
(43) Pub. Date: Dec. 21, 2023

(54) COGNITIVE REHABILITATION METHOD AND SYSTEM FOR ILLNESS-RELATED COGNITIVE IMPAIRMENT

(71) Applicant: Cognifit, Inc, San Francisco, CA (US)

(72) Inventor: Carlos Rodriguez, Madrid (ES)

(21) Appl. No.: 17/973,384

(22) Filed: Oct. 25, 2022

Related U.S. Application Data
(63) Continuation-in-part of application No. 17/842,051, filed on Jun. 16, 2022.

(51) Int. Cl. G16H 20/70; A61B 5/16

(52) U.S. Cl. CPC G16H 20/70; A61B 5/165

(57) ABSTRACT

A method and system are provided for the customized rehabilitation of cognitive abilities following an illness, such as COVID-19. The method comprises assessing the user's cognitive abilities by creating a post-illness cognitive impairment profile and, by presenting a stimulus and receiving a motion input, determining the skill level of a cognitive ability by presenting a stimulus and receiving a motion input. A customized precalculated training is then created and assigned to the user to rehabilitate the user's cognitive abilities. The precalculated training may comprise training games and assessment tasks specific to the user's illness and skill level and new games and tasks may be assigned as the user's skill level changes. This method may be implemented on a plurality of devices, and the responses may be transformed to uniformly compare the user's progress.

### GOVERNMENT CONTRACT

Not applicable.

### CROSS-REFERENCE TO RELATED APPLICATIONS

Pursuant to 35 U.S.C. § 120, this non-provisional patent application relies on the benefit of U.S. patent application Ser. No. 17/842,051 filed on Jun. 16, 2022. The content of said application is incorporated herein by reference in its entirety.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent documents or the patent disclosure, as it appears in the Patent and Trademark Office patent files or records, but otherwise reserves all copyrights and trade dress rights whatsoever.

### TECHNICAL FIELD

The disclosed subject matter relates generally to cognitive training, more particularly to a method and system for diagnosing the skill level of cognitive abilities and executing a customized rehabilitation program through a computing device.

### BACKGROUND

Illnesses are a common aspect of all individuals' lives, whether the flu, the common cold, or even more severe and even novel illnesses such as COVID-19. These illnesses often impact multiple aspects of an affected individual's life, through fatigue, nausea, congestion, fever, body aches, and headaches, among others. While many of these symptoms disappear after the illness, long-term symptoms may linger for days or even months. For example, 13% of COVID-19 patients experience long-term symptoms over a month after recovering from the initial illness. These long-term symptoms may include illness-related cognitive impairments such as, fatigue, difficulty thinking or concentration offered referred to as brain fog, headaches, sleep problems, depression, and anxiety, among others, which may prevent the individual from returning to normal life even after the illness has passed.

Treatment for these long-term symptoms may include rehabilitation services, however, such services can be expensive and time-consuming. Moreover, treatment of long-term symptoms is often postponed for some contagious illnesses, such as COVID-19, because prolonged periods of quarantine and/or isolation are required, even after initial symptoms fade. This leaves affected individual without the necessary treatment to fully recover from their illness. Further, doctor's appointments are notoriously expensive. While insurance may assist some, many insurances have high co-pays and may not cover the appropriate services. Therefore, there remains a need to increase the accessibility of rehabilitation treatment following illness-related cognitive impairment.

The applicant has proposed U.S. Pat. No. 6,632,174 to Breznitz to provide a method for testing and training cognitive abilities. However, the applicant has developed improvements since then which provide a customized rehabilitation program that is adapted to the user based on various factors to increase the program's efficacy. While his prior method taught that the steps may be customized for the individual users, it failed to provide illness-specific customization tailored to rehabilitate specific cognitive defects, longitudinal monitoring of the changes observed to provide a comparison to the desired progress, and oversight by a medical professional of a patient's progress. Indeed, technological changes have led to discrepancies in data collected from multiple devices, affecting the consistency and accuracy of the results. Thus, there is a need to standardize data from multiple devices.

Since Breznitz several methods to provide for evaluation and training of cognitive abilities have been proposed. For example, U.S. Pat. No. 7,347,818 to Simon, U.S. Application No. 2004/0,229,198 to Boyd, and U.S. Application No. 2006/0,252,014 to Simon. However, none of these proposals suggest a method to rehabilitate cognitive abilities following a cognitive impairment caused by illnesses. Instead, these methods merely test cognitive abilities and require an outside analysis of the test for diagnosis and treatment.

Another proposal, U.S. Application No. 2006/0,292,531 to Gibson, provides a method for developing learning skills and increased focus for students. However, Gibson's focus is the improvement of classroom skills and thus fails to consider the training of a full scope of cognitive abilities, including perception, linguistic, problem-solving, and motor skills. Further, there is no suggestion in Gibson that the results of the training may be sent to a third party, such as a medical professional or teacher, to monitor the results. While Gibson provides that the method may occur on an electronic device, it does not provide a method for uniformly comparing the collected data. Thus, this proposal fails to address the discrepancy in data collected across multiple electronic devices.

Thus, although various proposals have been made to evaluate and train cognitive abilities, none of those in existence provide a customized rehabilitation program operative to standardize data collected from a plurality of devices or otherwise combine the characteristics of the present invention.

### SUMMARY

The present disclosure is directed to a method and system, implemented on a computing device, for providing customized rehabilitation of cognitive abilities following illness-related cognitive impairment. An electronic device such as a tablet, laptop or desktop computer, or even a smartphone, may be operative to generate a user account comprising the user's sociodemographic information, illness information, and pre- and post-illness cognitive levels to determine a post-illness impairment targeted cognitive profile. A skill level may be determined for at least one cognitive ability by presenting at least one stimulus, receiving at least one motion input in response to the at least one stimulus, and analyzing at least one cognitive aspect associated with the at least one motion input. A precalculated training may be assigned to rehabilitate the user's skill level of at least one cognitive ability.

In accordance with one embodiment, the method and system allow rehabilitation of the cognitive skills following an illness. The illness may, for example only and not limitation, be any viral infection such as any of those caused by any influenza virus, rhinovirus, or corona virus, among others, whether known or novel; bacterial infections; autoimmune diseases such as lupus; and even mental illnesses, such as depression. A person of ordinary skill will appreciate the method and system may be utilized with any form of cognitive impairment and the aforementioned illnesses are for example only. In the interest of brevity, the illness is referred to with reference to a recent novel corona virus known to cause long-term cognitive impairment, COVID-19, however, any illness may form the basis for treatment via the cognitive rehabilitation method and system for illness-related cognitive impairment.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In order to identify the user, the system is operative to receive biographical information including, but not limited to the user's name, date of birth, billing information, and contact information such as an email address or even a physical address. A person of ordinary skill in the art will recognize other forms of biographical information exist and may be used in practicing the invention. In addition, the system may be operative to assign a user account to the biographical information.

A post-illness cognitive impairment battery may be conducted and information relating to at least one targeted domain may be received. Each of the at least one targeted domains may comprise information relating to the user's sociodemographic factors, illness, pre- and post-illness cognitive levels.

The sociodemographic factors may be received from an assessment of the user's basic descriptive data. This data may, for example, and without limitation comprise the user's age, gender, and country. In some embodiments, the sociodemographic factors may comprise the user's marital status, living arrangement, education status, employment status, and combinations thereof. A person of ordinary skill in the art will appreciate the aforementioned sociodemographic factors is provided for example only and any sociodemographic factors known in the art may be utilized.

The illness information may comprise a user's medical history. For example, the user's medical history may comprise information regarding a type of illness, a time when the illness occurred, a severity of the illness, and a duration of the illness. In one embodiment, the illness may be COVID-19 and a COVID-19-specific assessment may be conducted. The COVID-19-specific assessment may determine the type and sub-type of the illness-related cognitive impairment. More particularly, the COVID-19 specific assessment may determine a type of severity of post-acute COVID-19 syndrome (PACS). Post-acute COVID-19 syndrome (PACS) is well-known to a person of ordinary skill in the art. In some embodiments, the COVID-19 specific assessment may be a conventional assessment in the field. For example, the COVID-19 specific assessment may be a conventional assessment to analyze the effects of the illness, such as the Cognitive Change Questionnaire, Patient-Reported Outcomes Measurement Information System (PROMIS), WHOQOL, and/or Montreal Cognitive Assessment Test (MoCA). However, a person of ordinary skill will appreciate that illness information may be extracted from any conventional assessment or unconventional assessment known in the art.

For example, and without limitation, the illness information may comprise information relating to the user's oxygen levels, respiratory rate, and/or medical intervention. In one embodiment, the illness information may comprise information relating the user's potentially affected systems of the user. Such as, and without limitation to, general respiratory systems, cardiovascular systems, neuropsychiatric or neuropsychological systems, dermatological systems, and/or gastrointestinal systems.

The information relating to the user's pre-illness cognitive level may be extracted from a pre-illness cognitive assessment. The pre-illness cognitive assessment may include information regarding the user's global physical health prior to the illness. In one embodiment, the pre-illness cognitive assessment may be a subjective questionnaire operative to collect a self-reported status of at least one skill level of at least one cognitive ability of the user prior to the illness. In some embodiments, the pre-illness cognitive assessment may be displayed to the user. However, in yet another embodiment, the pre-illness cognitive assessment may be an assessment conducted by a third party or system and may be received by the system.

The at least one cognitive ability may, for example, and without limitation, be related to memory, attention, and/or executive function. More particularly, the at least one cognitive ability may be related to perception, focused attention, divided attention, short and long-term memory, linguistics, decision making, problem-solving, psycho-motor abilities, and meta-cognitive abilities. A person of ordinary skill will appreciate the aforementioned cognitive abilities are not an exhaustive list and all cognitive abilities are available.

It is contemplated that because the method and system are for rehabilitation, the user may not create the user account until the illness has occurred. Thus, it is contemplated that the pre-illness information may be a subjective belief of the user's skill level prior to the illness. In some embodiments, the user may report their subjective belief of their skill level prior to the illness. In another embodiment however, the third party may report their subjective belief of the user's illness level prior to the illness. It is contemplated that in such an embodiment, familiarity with the user pre-illness may be collected in addition to the subjective belief from the third party. Thus, the third party may be any of a family member, friend, acquaintance, medical provider, or caretaker of the user, or any other third party having some knowledge of the user's pre-illness skill level. However, in some embodiments, the user account may be created prior to the illness and thus the pre-illness information may comprise objective information from assessments prior to the illness. It is further contemplated that the pre-illness information may comprise a combination of subjective and objective information regarding the user prior to the illness.

The information relating to the user's post-illness cognitive level may be extracted from a post-illness cognitive assessment operative to collect information about the user following the illness. The post-illness cognitive assessment may, for example, and without limitation, comprise information on the user's quality of life following the illness and one or more cognitive domains.

The one or more cognitive domains may, for example, comprise an attention domain, a memory domain, an executive function domain, or any combination thereof.

The attention domain may comprise cognitive abilities such as the user's divided attention, focused attention, response time, processing speed, and visual scanning skills. It is further contemplated that the one or more cognitive domains may, without limitation, comprise a memory domain. The memory domain may comprise recognition, phonological short-term memory, contextual memory, non-verbal memory, visual short-term memory, and naming skills. The one or more cognitive domains may further comprise an executive function domain. The executive function domain may comprise skills relating to inhibition, updating, planning, shifting, and working memory. For example, the executive function domain may be any of recognition, phonological short-term memory, contextual memory, short-term memory, non-verbal memory, visual short-term memory, naming, divided attention, focused attention, response time, processing speed, visual scanning, inhibition, updating, planning, shifting, and working memory. It is contemplated that the one or more cognitive domains may comprise the aforementioned cognitive domains alone or in combination, however, a person of ordinary skill will recognize that all cognitive domains known in the art may be utilized.

In a further embodiment, the one or more cognitive domains may comprise an assessment of the at least one cognitive ability associated with the specific cognitive domain. The assessment of the skills may allow for the information received from the post-illness cognitive assessment to be uniformly compared to the same one or more cognitive domains located in a database. The database may store information from a plurality of users. Of the information stored in the database, information from the plurality of users having the same or similar illness may be compared to the user's information received from the post-illness cognitive assessment. For example, a user whose illness-related cognitive impairment is related to COVID-19 illness may be compared to other users who suffered from COVID-19 illness. More particularly, the user may be compared to other users who have suffered from a similar COVID-19 illness, such as, and without limitation to, the duration, severity, and intervention. It is well understood that each person may experience COVID-19 differently and as a result may experience varying levels of cognitive impairment. COVID-19 related cognitive impairment may result from complications associated with COVID-19 such as sepsis, hypoxia, immune hyperstimulation, cerebrospinal fluid antibodies, white matter change, or any of a wide variety of COVID-19 related complications. A person of ordinary skill in the art will recognize that such complications may result in any of a stroke, encephalopathies, inflammatory syndrome, microbleeds, and autoimmune responses, among others. These complications may result in injuries to a specific region of the brain, thus, causing impairment to different cognitive abilities. Therefore, comparing users having the same or similar illness may allow for the comparison of illnesses that effect the same region of the brain and thus the same or similar cognitive abilities may be affected.

The database may further comprise normative data of the one or more cognitive domains representing an expected skill level of the at least one cognitive ability in the one or more cognitive domains. In some embodiments, the normative data may be calculated for each user based on the user's provided information. For example, and without limitation, the normative data may be dependent on the type of illness suffered by the user, the post-illness quality of life, sociodemographic status, and/or the biographical information. In another embodiment, the normative data may be uniform for all users, without consideration to the provided information. It is contemplated that the normative data may be continually updated each time a new user's information is received by the database, thus allowing the system to become more accurate over time.

The information received in the post-illness cognitive impairment battery may inform a post-illness cognitive impairment profile. The post-illness cognitive impairment profile may be user-specific. As no two individuals are identical and illness effect each individual differently, it is contemplated that no two each post-illness cognitive impairment profile may be identical.

The skill level of at least one cognitive ability may be determined by presenting at least one stimulus, receiving at least one motion input, and analyzing at least one cognitive aspect associated with the at least one motion input. In some embodiments, the step of presenting the at least one stimulus may occur on the computing device. The step of presenting the at least one stimulus may comprise the step of changing the stimulus. Changing the stimulus may comprise, for example, moving the location of the stimulus on a display screen, changing the color of the stimulus, changing the shape of the stimulus, or combinations thereof. A person of ordinary skill in the art will appreciate that the aforementioned changes to the stimulus are provided for example only and any changes in the stimulus may be utilized to practice the invention. In some embodiments, the at least stimulus may simultaneously target multiple senses, such as vision and hearing. However, in another embodiment, the at least one stimulus presented may target only one sense.

In some embodiments, the at least one stimulus may be at least one abstract stimulus, at least one meaningful stimulus, or a combination thereof. The at least one abstract stimulus may, for example, and without limitation, be at least one visual stimulus, such as a color or shape, displayed on the computing device. The at least one meaningful stimulus may, for example, comprise a known fact or feature that the user may memorize or identify. For example, the at least one meaningful stimulus may be a pattern displayed on the computing device. A person of ordinary skill in the art will appreciate the at least one stimulus described are for example only and any stimulus known in the art is available to practice the invention.

At least one motion input may be received by the computing device in response to the at least one stimulus. In some embodiments, the at least one motion input may be operative to move the at least one stimulus, position the at least one stimulus, move a peripheral device to intercept the at least one stimulus, operate a control with discrete states, or adjust the stimulus. A person of ordinary skill in the art will appreciate that the aforementioned operations of the motion inputs are provided for example only and any motion input known in the art may be utilized.

It is contemplated that the at least one motion input received may depend on the computing device. For example, the at least one motion input received from a personal computer may be received through a mouse or keypad. In another example, the at least one motion input received from a tablet computer or smartphone may be a touch input received through a touchscreen display. In yet another example, the at least one motion input received from a laptop computer may be received from a keyboard or a trackpad. A person of ordinary skill will appreciate that the aforementioned methods of receiving the at least one motion input are provided as examples only and any input known in the art to be received by the computing device may be utilized.

The at least one motion input may be received on the computing device and sent to a database. In one embodiment, the database may comprise the at least one motion input from the user. In another embodiment, the database may comprise the at least one motion input received from the plurality of users. In either such embodiment, the at least one motion input received from the user may be validated before being stored in the database. The validation may comprise determining whether the at least one motion input is a false negative or a false positive, preventing the recordation of outliers, and other validation known in the art. For the purpose of illustration, the false positive may be a correct motion input in response to the at least one stimulus that was made through accident or error, such as a guess or accidental movement of the mouse. In another example, the false negative may be an incorrect motion input in response to the at least one stimulus that was made in error, such as a glitch on the computing device wherein, if the glitch had not occurred, the correct motion input would have been received. If the at least one motion input cannot be validated it may not be stored in the database and the steps of presenting at least one stimulus and receiving at least one motion input may be repeated.

The step of analyzing at least one cognitive aspect may be configured to determine from the at least one motion input the skill level of at least one cognitive ability. In some embodiments, the step of analyzing the at least one cognitive aspect may comprise a test of the user's preliminary cognitive levels. The test may comprise displaying a series of assessments and/or trainings and generating at least one result in response to the series of assessments and/or trainings. The at least one result may be broken into discrete results for each of the cognitive aspects tested and the skill level for each of the cognitive abilities associated with the cognitive aspect may be determined.

In another embodiment, the step of analyzing the at least one cognitive aspect may comprise creating a testing task operative to test one cognitive ability, assign the testing task to the user, receive a response to the testing task, and create additional testing tasks as necessary. It is contemplated that each testing task created may be operative to test a specific cognitive ability. In some instances, multiple testing tasks may be created to test different cognitive aspects of the same cognitive ability. Once the testing task is completed and there are no additional cognitive abilities to test, no further testing tasks are necessary, and the skill level of the at least one cognitive ability may be determined. In some such embodiments, the testing task may be the same for all users, regardless of the user's post-illness cognitive impairment profile. However, in another embodiment, the testing task may be assigned based on the user's post-illness cognitive impairment profile. In some embodiments, the cognitive skill level of the at least one cognitive ability may be determined by the results of each of the testing tasks. However, in another embodiment, the cognitive skill level of the at least one cognitive ability may be determined by the results from at least one testing task.

The precalculated training may be operative to rehabilitate at least one cognitive ability. In one embodiment, the at least one cognitive ability to be rehabilitated may be determined by the skill level of the at least one cognitive ability. In some such embodiments, the precalculated training may target the at least one cognitive ability having the lowest skill level. In another such embodiment, the precalculated training may target the at least one cognitive ability having any skill level. In yet a further such embodiment, the precalculated training may target the at least one cognitive ability in an order calculated to reduce user frustration. It is contemplated that the precalculated training may target the at least one cognitive ability as a whole or as discrete aspects of the at least one cognitive ability.

In some embodiments, the precalculated training may comprise at least one training game and at least one assessment task. For example, the precalculated training may comprise two training games and one assessment task. However, any number of training games and assessment tests may comprise the precalculated training.

The at least one training game in the precalculated training may be selected from a plurality of training games. In some embodiments, the plurality of training games may be arranged in a sequence, however, the plurality of training games may be stored in a randomly accessible format. When the plurality of training games is organized in a sequence, at least one training game may be in any order. For example, the plurality of training games may be ordered in the sequence depending on the complexity, name, time necessary for completion, compatibility with computing devices, length of time in sequence, at random, or in any other order known in the art.

The first training game in the sequence of the plurality of training games may be loaded. When the first training game in the sequence has not been completed, an available status may be assigned to the training game, and the next training game in the sequence may be loaded. If the first training game in the sequence has been completed, no further action may be taken with the first training game, and the next training game in the sequence may be loaded. These steps may be repeated for each of the plurality of training games in the sequence.

Once each of the plurality of training game has been loaded, the number of available training games is returned. If the number of available training games is greater than a predetermined number of training games, a training database and the user's history may be loaded. The set of training games may comprise a predetermined number of the available training games, which, itself, comprises the at least one training game assigned to the user for the precalculated training. For example, and without limitation, the predetermined number may be one, two, three, four, five, ten, fifteen, or twenty or any other predetermined number as needed or desired.

The training database may comprise information, including, without limitation, complexity, targeted cognitive abilities, estimated time, and compatibility with the computing device, relating to each of the available training games. The training database may further comprise information relating to other users in the database and known medical treatments and methods. Such information may comprise, for instance, each user's history comprising, as nonlimiting examples, the user's biographical information, post-illness cognitive impairment profile, skill levels of at least one cognitive ability, previously assigned pre-calculated trainings, and previous results from at least one training game.

Next, the weight of each available training game may be calculated to control which of the available training games is assigned. In some embodiments, the weight of each available training game may be calculated by the results of a previous at least one training game loaded from the user history. However, in another embodiment, the weight of each available training game may be calculated by the information loaded from the training database, including, without limitation, results from a plurality of users, complexity, and the targeted cognitive ability.

In yet a further embodiment, the weight of each available training game may be calculated by information loaded from the training database and the user history. For example, the weight may be calculated based on a variety of factors, such as, the user's results from previous at least one training task and at least one assessment task, the results from previous at least one training task and at least one assessment task from user's having the same or similar illness, the complexity of the tasks, or any other factor contemplated in the art. In an exemplary embodiment, the weight may be calculated based on a relation between the skill level of the at least one cognitive ability and the at one cognitive ability targeted by the pre-calculated training. However, in another embodiment, the weight may be a pre-determined.

In some embodiments, the weight may identify the complexity of each available training game and may assign the least complex of the available training games. However, in one embodiment, the most complex of the available training games may be assigned. In yet a further embodiment, the available training games assigned may comprise a complexity similar to the skill level of the user's at least one cognitive ability.

In another embodiment, the weight may be calculated to identify which of the available training games may advance the user's rehabilitation while reducing frustration. In other words, the available training games where the user is likely to find a high level of success may be weighted more highly than those available training games where the user is likely to experience a low level of success. A person of ordinary skill will appreciate the aforementioned weights are provided as examples only, and the weight may be determined by any factor, or combination of factors, known in the art.

However, if the available games are less than the predetermined number of the available training games, all available training games may be assigned. Thus, in some instances, the assigned number of available training games may be less than the predetermined number of available training games. As a clarifying example, when the predetermined number of available training games is two and there is only one available training game, only the one available training game may be assigned.

The assigned training games may be saved to the user account. In some embodiments, the assigned training games may be saved to the training database. Thus, the assigned training games may be available on any computing device operative to access the user account.

A sequence of assessment tasks operative to gauge the skill level of at least one cognitive skill targeted by the assigned training games may be generated. The first assessment task in the sequence of assessment tasks may be loaded and the completion status of the assessment class may be returned. If the first assessment task in the sequence has not been completed then the assessment task may be assigned. However, if the first assessment task has not been completed and there are more assessment tasks in the sequence, the next assessment task may be loaded and may follow the same process as the first assessment task. The process of loading the next assessment task in the sequence may be repeated until the loaded assessment task is not completed and may be assigned. If all assessment tasks in the system are completed, the assessment task having the oldest date of completion may be assigned to the user.

The assigned training games and the assigned assessment task may be returned. The returned training games and assessment task may comprise a custom selection of the at least one training game and at least one assessment task operative to rehabilitate the user's specific cognitive deficiencies as assessed from the skill level of the one or more cognitive abilities.

The pre-calculated training may comprise the assigned training games and the assigned assessment task, and thus may be unique to each user. Further, the pre-calculated training may be updated and may comprise newly assigned training games and/or assessment tasks. Thus, the process of loading and assigning the at least one training game and the at least one assessment task may be repeated.

In one embodiment, the pre-calculated training may be performed by a computerized cognitive training tailored to the user account. The computerized cognitive training is a computer program, for example, a digital application, comprising the pre-calculated training. In some such embodiments, the pre-calculated training may be gamified, engaging, rehabilitation tasks accessible from the computer program. It is contemplated that by providing computerized cognitive training the user's access to the pre-calculated training may increase. Thus, allowing the user to complete the pre-calculated training in any location, such as, and without limitation, the user's home, gym, or medical office. It is further contemplated that utilizing the computerized cognitive training may increase engagement over traditional rehabilitation methods as the computerized cognitive training may be presented in a game-like manner, increasing enjoyment while reducing frustration.

The computerized cognitive training may be accessible across a plurality of computing devices. For example, the computerized cognitive training may be accessible on a personal computer, smartphone, television, tablet computer, laptop computer, and virtual reality headset. A person of ordinary skill in the art will appreciate the aforementioned computing devices are for example only and without limitation. It is contemplated that a plurality of the computing devices may be utilized. However, a response to the pre-calculated training received from computing devices may comprise inherent differences, such as differences in reaction time and accuracy. For example, the results collected on touchscreen devices, such as tablets or smartphones, may vary in accuracy from a mouse-enabled device, such as personal computers, because of the differences in the interface. Thus, in some embodiments, the responses received on the computing devices may be transformed to create uniform results from the plurality of computing devices. For example, and without limitation, the response received from the computing device may be transformed such that the response may be uniform to the response received on the personal computer. It is contemplated that by transforming the results, the user's progress may be better understood and the results collected on a plurality of computing devices may be uniformly compared and progress may be accurately tracked.

It is contemplated that in some embodiments, the system may be operative to continually update the information stored in the database. By continually updating the information, the system may become more accurate over time and better reflect the user, thus allowing for increased rehabilitation. This may be accomplished by reducing the impact of outliers in the system and providing more data. A person of ordinary skill in the art will appreciate how the presence of additional data points may increase the accuracy of the system. In some embodiments, the updating of the information stored in the database may occur after the steps of determining the skill level and assigning the precalculated training.

In some embodiments, the user account may be accessed by a third party, such as a medical professional, caregiver, family member, or insurance company, to share the user's progress to be tracked. It is contemplated this may increase compliance with the rehabilitation program

In embodiments where a medical professional has access to the user account, the system may be operative to receive an instruction from the medical professional. In response to the instruction, the system may save information to the user account, assign one or more rehabilitation tasks, or display a message to the user. A person of ordinary skill will appreciate that the provided system responses are provided for example only and are not limited to those listed.

Several advantages of one or more aspects of the method and system for customizable cognitive rehabilitation are that they:
a.) increase accessibility to cognitive rehabilitation programs;
b.) provide customizable rehabilitation following illness-related cognitive impairment;
c.) continuously customize trainings assigned to the user to promote recovery;
d.) reduce the time commitment for cognitive rehabilitation;
e.) transform results received from different computing devices to allow for uniform comparison of results collected from multiple computing devices; and
e.) provide progress updates to medical professionals for oversight.

Thus, it is an object of this method and system to provide access to cognitive rehabilitation through a custom training. It is a further object of this method and system to provide a diagnosis of the skill level of at least one cognitive ability and assign training designed to improve the skill level of at least one cognitive ability.

It is another object of this method and system that the user may access the cognitive rehabilitation program on a plurality of computing devices. It is yet another object of this method and system that the results received on the plurality of computing devices may be transformed for uniform comparison of the results.

It is an object of this system and method to promote the cognitive health of the user following illness-related cognitive impairment.

It is still another object of this system and method to be used in accordance with medical professionals.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1A is a blocked diagram of a networked environment in which an exemplary embodiment of a system for customized cognitive rehabilitation is implemented.

FIG. 1B is a flowchart depicting an exemplary embodiment of a method for customizable cognitive rehabilitation may be performed by the system shown in FIG. 1A.

FIG. 2 is a flowchart depicting an exemplary embodiment of one aspect of a method for cognitive rehabilitation in accordance with one embodiment, namely determining the skill level of at least one cognitive ability.

FIG. 3 is a flowchart depicting another exemplary embodiment of one aspect of a method for cognitive rehabilitation in accordance with one embodiment, namely determining the skill level of at least one cognitive ability.

FIG. 4 shows an exemplary embodiment of a computing device shown in FIG. 1A.

FIG. 5A-B are flowcharts depicting one exemplary aspect of the method for customizable cognitive rehabilitation in accordance with one embodiment, namely, determining a pre-calculated training.

FIG. 6A-B are flowcharts depicting one exemplary aspect of the method for customizable cognitive rehabilitation in accordance with one embodiment, namely, calculating the skill level of at least one cognitive ability to determine a new pre-calculated training.

FIG. 7 is a flowchart depicting one exemplary aspect of the method for customizable cognitive rehabilitation in accordance with one embodiment, namely, calculating the skill level of at least one cognitive ability.

The disclosed embodiments may be better understood by referring to the figures in the attached drawings, as provided below. The attached figures are provided as non-limiting examples for providing an enabling description of the method and system claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

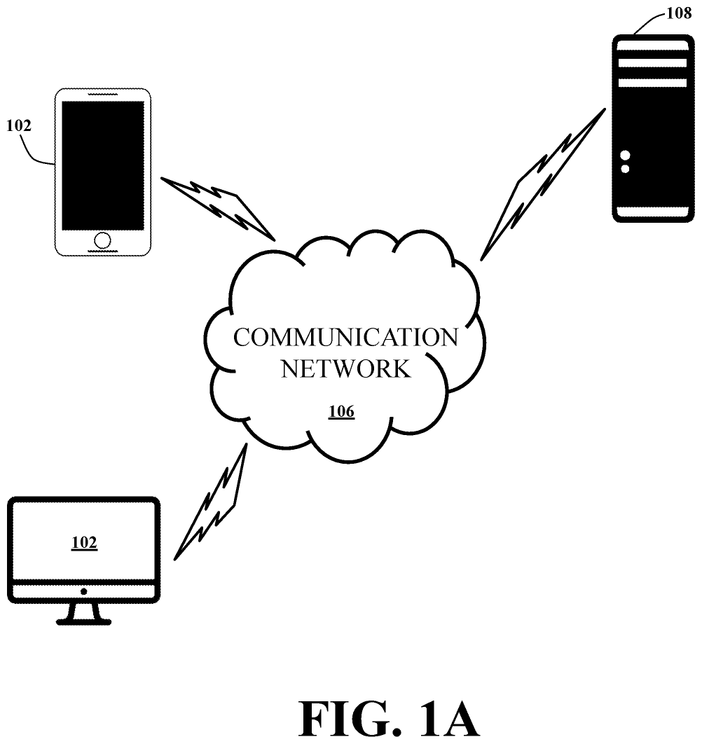

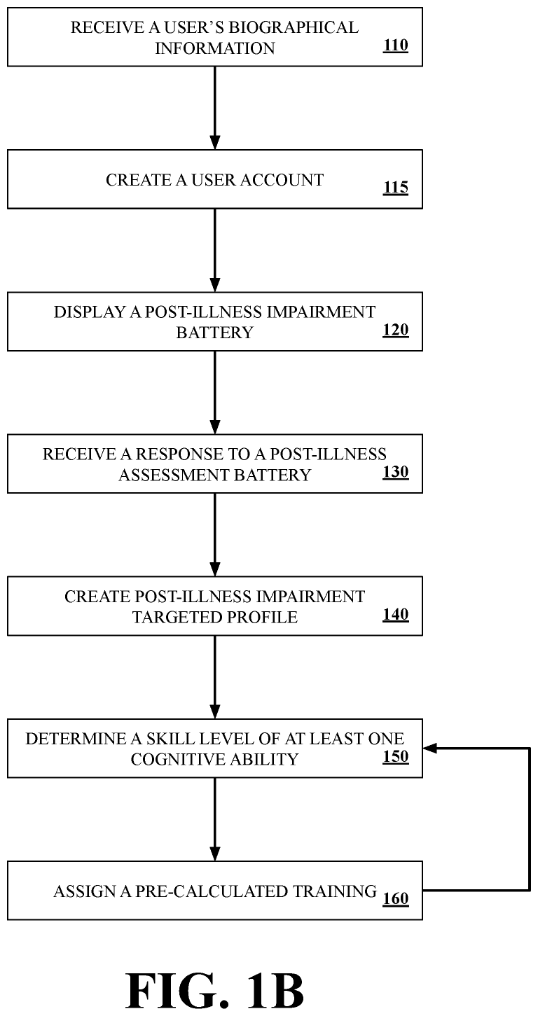

A description of an embodiment of a method and system for customizable cognitive rehabilitation is now described followed by a discussion of the operation of various components within the system. In this regard, FIG. 1A illustrates an exemplary embodiment of the system for customizable cognitive rehabilitation 100 which includes at least one computing device 102 and 104 coupled to a database 108. FIG. 1A illustrates two computing devices 102 and 104 coupled via a communication network 106. Each of the computing devices may be embodied as a personal computer, tablet computer, laptop computer, or smartphone. Notably, the communication network can use one or more various communication types such as, for example and without limitation, cellular and Wi-Fi communications.

A user of the at least one computing devices 102 and 104 may use their devices to access the system 100 for customizable cognitive rehabilitation. In one embodiment, the user may access the system 100 on a first computing device 102 being a personal computer. In one embodiment, a third party may use the at least one computing device 102 and 104 to interact with the user. The third party may, as a non-limiting example, be a medical professional, care-giver, family member, or insurance agency.

In this regard, a first computing device 102 includes the system 100 for customizable cognitive rehabilitation, which can be implemented in numerous ways such as, for example and without limitation, a downloadable application, web-based application, website, or the like.

With reference to FIG. 1B an exemplary method and system 100 for customizing cognitive rehabilitation is provided. The method includes the steps of: receiving a user's biographical information 110; creating a user account 115; displaying a post-illness impairment battery 120, receiving a response to a post-illness impairment battery 130; creating a post-illness impairment targeted profile 140; determining a skill level of at least one cognitive ability 150; and assigning a pre-calculated training 160.

The user's biographical information may comprise information relating to the user's name, contact information, date of birth, billing information, or combinations thereof. The biographical information may further comprise a referral for cognitive rehabilitation from a medical professional, such as a doctor or physical therapist. A person of ordinary skill will appreciate the aforementioned biographical information is provided as an example only and all biographical information known in the art may be received.

The biological information may be received 110 through user input or by extracting the data from another source, such as the referral or medical records.

The step of creating the user account 115 may utilize the biographical information received in the aforementioned step 110 to create the account. The biographical information may be assigned to a user identifier. For example, the user identifier may be the user's own name or email address, or any other username known to those of ordinary skill in the art, such as any unique series of numbers, letters, or combination of numbers and letters as selected by or assigned to the user. In some embodiments, the user identifier may comprise any digital key known to those of ordinary skill in the art. This may be particularly desirable for patients suffering from memory loss that could prevent them from recalling their own identifier on demand. The user identifier may identify the specific user on a variety of computing devices as shown in FIG. 1A. Further, it is contemplated that in some embodiments, the third party may access the user account through the user identifier.

Returning to FIG. 1B, the information received in response to the post-illness cognitive impairment battery 130 may comprise information relating to the user's illness, sociodemographic factors, and pre- and post-illness cognitive levels. The information relating to the user's illness may comprise information regarding a type of illness the user suffered and information relating to the occurrence of the illness. In one embodiment, the type of illness may be a clinical diagnosis of the illness. It is contemplated that the clinical diagnosis may be received from the user or from the medical professional, either directly or through a referral. In one exemplary example, the illness may be COVID-19 and the illness information may comprise information relating to the user's potentially affected systems. Such as, and without limitation to, general respiratory systems, cardiovascular systems, neuropsychiatric or neuropsychological systems, dermatological systems, and/or gastrointestinal systems.

Further, the illness information may relate to any of an occurrence of the illness, including, as a non-limiting example, when the illness occurred, the severity of the illness, cause of the illness, and interventions that were taken at the time of the illness. A person of ordinary skill will appreciate other information relating to the illness that may be utilized.

The information relating to the user's illness may comprise information relating to the user's cognitive abilities following the illness. For example, and without limitation, such information may be related to any of depression, anxiety, insomnia, fatigue, and brain fog, among others.

The user's sociodemographic factors may comprise information relating to the user's age, gender, and country. The sociodemographic factors may further comprise information relating to marital status, living arrangement, education level, and employment status. A person of ordinary skill in the art will recognize other forms of sociodemographic information.

The pre-illness cognitive level may comprise information relating to a skill level of at least one cognitive ability prior to the illness. Additionally, the pre-illness cognitive level may comprise information regarding the user's global physical health prior to the illness. In one embodiment, the pre-illness cognitive level may be extracted from a pre-illness cognitive assessment.

In an exemplary embodiment, the pre-illness cognitive levels may be collected following the illness and represent the user's subjective assessment of the skill level of at least one cognitive ability before the illness. However, in some embodiments, the pre-illness cognitive level may be determined by an assessment that occurred prior to the illness and thus be an objective assessment. It is contemplated that the assessment may be a general assessment and the skill level of at least one cognitive ability may be extracted from the general assessment. Some embodiments may utilize a combination of subjective and objective assessments. In such an embodiment, the subjective assessment may be utilized to supplement the information received from the objective assessment. However, in some embodiments, the subjective and objective assessments may be utilized for the same cognitive ability and may have different weights from one another.

The post-illness cognitive level may be related to the skill level of at least one cognitive ability following the illness. For example, the post-illness cognitive level may comprise information on the user's quality of life following the illness and cognitive functioning in one or more cognitive domains.

The one or more cognitive domains may be an attention domain, memory domain, executive function domain, or a combination thereof. More particularly, the attention domain may comprise at least one cognitive ability relating to attention. For example, the at least one cognitive ability relating to attention may comprise divided attention, focused attention, response time, processing speed, and visual scanning. However, a person of ordinary skill in the art will recognize other cognitive abilities relating to attention. The memory domain may comprise at least one cognitive ability relating to memory, including, for example, recognition, phonological short-term, contextual, non-verbal, visual short-term, and naming memory skills. Of course, a person of ordinary skill in the art will recognize other cognitive abilities relating to memory. The executive function cognitive domain may comprise cognitive abilities such as inhibition, updating, planning, shifting, and working memory, however, still, a person of ordinary skill will appreciate other cognitive abilities relating to executive function. Thus, the forgoing for each cognitive domain is offered for the purposes of example only and not limitation.

Each of the one or more cognitive domains may comprise at least one cognitive aspect. The at least one cognitive aspect are contemplated to be discrete components of the at least one cognitive ability in the cognitive domain. As a clarifying example, and without limitation, the at least one cognitive aspect in the executive function domain may be cognitive flexibility, theory of mind, anticipation, emotional self-regulation, and sequencing.

In some exemplary embodiments, the one or more cognitive domains may comprise an assessment of the skill level of at least one cognitive ability. It is contemplated that the assessment may allow the collected information to be uniformly compared to the same one or more cognitive domains stored in the database 108 shown in FIG. 1A.

Continuing with FIG. 1B, the post-illness impairment profile (referenced in block 140) may comprise the information received from the post-illness cognitive impairment battery 130 as compared to a dataset representing an expected cognitive functioning in one or more cognitive domains for a person with similar sociodemographic factors and illness. The post-illness impairment profile may indicate at least one cognitive domain that may be improved with customized rehabilitation. In some embodiments, the post-illness impairment profile may compare the user's cognitive functioning in one or more cognitive domains to the cognitive functioning in one or more cognitive domains of individuals with a similar illness.

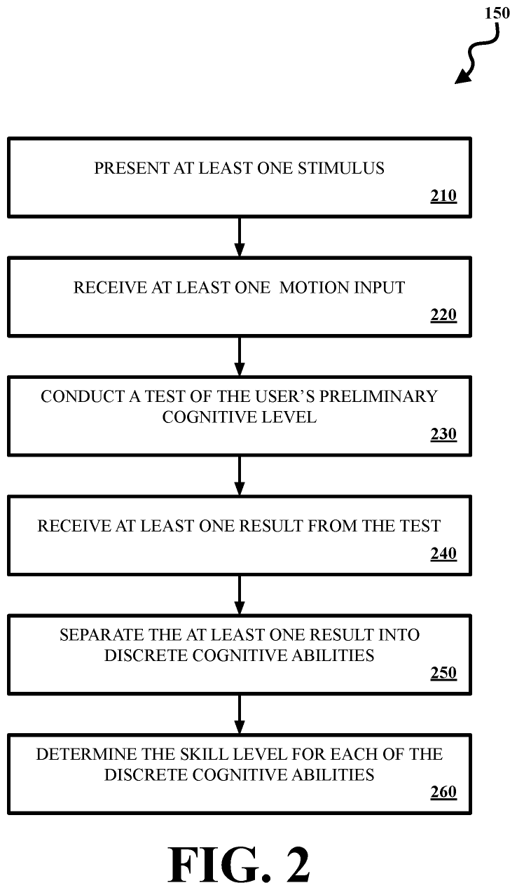

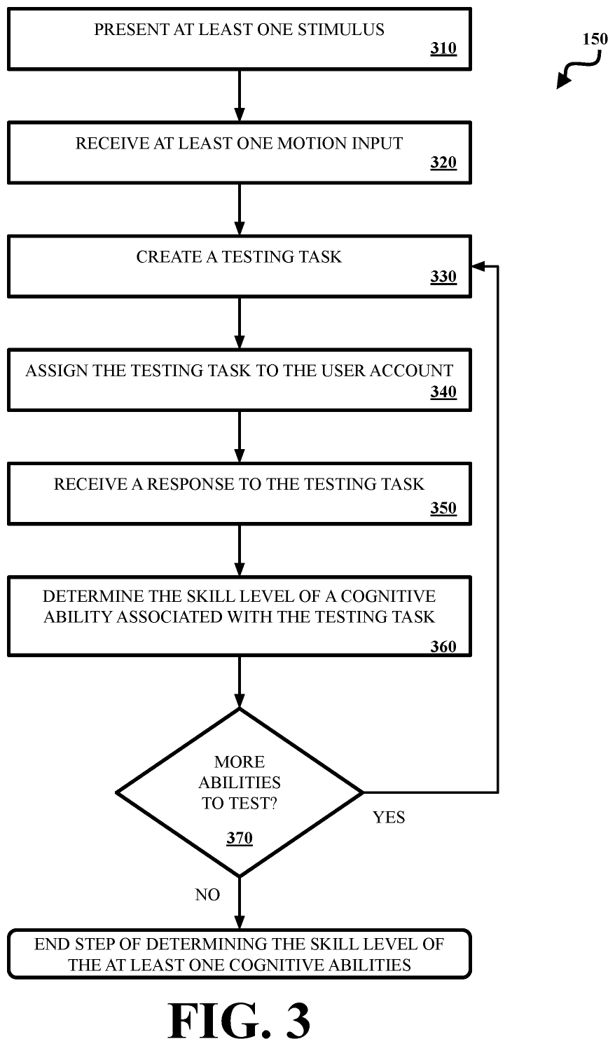

The step of determining a skill level of at least one cognitive ability 150 may itself comprise the steps shown in FIGS. 2-3 and be carried out on the at least one computing device 102 and 104 in FIG. 1A. As shown in FIG. 2, one embodiment for determining the skill level of at least one cognitive ability may comprise the steps of: presenting at least one stimulus 210; receiving at least one motion input 220; conducting a test of the user's preliminary cognitive level 230; receiving at least one result from the test 240; separating the at least one result into discrete cognitive abilities 250; and determining the skill level for each of the discrete cognitive abilities 260.

Another embodiment of determining the skill level of at least one cognitive ability 150 is shown in FIG. 3. The step of determining the skill level may comprise the steps of: presenting at least one stimulus 310; receiving at least one motion input 320; creating a testing task 330; assigning the testing task to the user account 340; receiving a response to the testing task 350; and determining the skill level of one cognitive ability associated with the testing task 360. The testing task may target only one cognitive ability and the steps of creating the testing task 330, assigning the testing task to the user account 340, receiving the response to the testing task 350, and determining the skill level of one cognitive ability associated with the testing task 360 may be repeated until all desired cognitive abilities are tested. In some embodiments, the skill level of the previously tested cognitive abilities 330 may determine the next testing task 330. Further, the responses to the testing task 350 may determine whether more testing tasks 330 need to be created. In some embodiments, each of testing tasks 350 may determine the skill level of one cognitive ability 360. However, in other embodiments, multiple testing tasks 350 may be created to determine the skill level of one cognitive ability 360 and the testing tasks 350 may be operative to test discrete cognitive aspects of the cognitive ability.

The at least one stimulus 210 and 310 of FIGS. 2 and 3 may be at least one abstract stimulus, at least one meaningful stimulus, or combinations thereof. It is contemplated that the at least one stimulus 210 and 310 may be displayed on the at least one computing device 102 and 104 in FIG. 1A. The at least one abstract stimulus may, for example, be at least one observable visual stimulus. In some embodiments, the at least one abstract stimulus may comprise one or more shapes. It is contemplated that the one or more shapes of the at least one abstract stimulus may be inconsequential in determining the skill level of the one or more cognitive abilities. However, in some embodiments, the one or more shapes of the at least one abstract stimulus may instead be consequential in determining the skill level of the one or more cognitive abilities.

The one or more meaningful stimulus may, as a non-limiting example, comprise a known fact or feature. For example, the known fact may comprise presenting a common object, such as a cup or bowl, and a series of letters comprising at least the letters necessary to spell the displayed object. In a further example, the known fact may comprise presenting a pattern on a series of displayed objects.

The step of presenting the at least one exemplary FIG. 2 or FIG. 3 stimulus 210 and 310 may further comprise a step of changing any of the at least one stimulus. For example, the at least one stimulus may move from a first to a second location on a display screen. In a further example, the at least one stimulus may change from a circle to a square, however, it is contemplated that the aforementioned shapes are for example only and any shape may be used. Any changes at all may be made to the at least one stimulus to practice the invention, and in a further non-limiting example these changes may be any change in position, shape, size, color, speed, or visibility.

Receiving at least one motion input as in FIGS. 2 and 3 (220, 320) may comprise a response to the one or more stimulus presented. For example, the at least one motion input 220 and 320 may comprise moving the at least one stimulus, operating a control with discrete states, adjusting the at least one stimulus, and selecting any of the at least one stimulus. However, a person of ordinary skill in the art will appreciate that the provided examples are for example only, and any motion input may be utilized. Continuing with a previous example wherein the at least one stimulus is the presenting of common objects and series of letters, the at least one motion input may comprise receiving a selection of the letters necessary to spell the name of the object. In a further continuation of a previous example wherein the stimulus is the presentation of the pattern, the at least one motion input may be the repetition of the pattern.

In one exemplary embodiment, the steps of determining the skill level of at least one cognitive ability 150 may utilize methods known in the art. For example, methods from known neurological assessments or tasks such as Tower of London, Hooper Visual Organization Task™, Continuous Performance Test, Test of Memory Malingering, Test of Variables of Attention, and Wisconsin Card Sorting Test may be utilized. A person of ordinary skill in the art will appreciate that the aforementioned methods are for example only and not limitation. In some embodiments, the aforementioned methods may be utilized in their classic form. However, in other embodiments, the aforementioned methods may be transformed and/or combined to create unique tests that may comprise common steps.

The step of receiving at least one motion input 220 and 320 may be received on the at least one computing device 102 and 104 in FIG. 1A. Returning to FIGS. 220 and 320, in some embodiments, the at least one motion input 220 and 320 may be received through a mouse operative to control a cursor displayed with the one or more stimulus. In another embodiment, the at least one motion input may be received from a keyboard wherein the at least one motion input may be the selection of one or more keys. Any known method of receiving motion input on the computing devices may be utilized including, as a further example, input from a touchscreen, trackpad, camera, or audio-device.

With particular reference to FIG. 2, the next step of determining a skill level of at least one cognitive ability 150 may comprise conducting a test of the user's preliminary cognitive level 230. The test may comprise a series of assessments or trainings that are operative to determine the skill level of at least one cognitive ability. Receiving at least one result 240 may comprise receiving at least one response from the user that correlates with at least one cognitive aspect of at least one cognitive ability. The at least one result may be broken into a discrete result for each of the cognitive aspects tested. and the skill level of each of the discrete cognitive abilities may be determined 250.

As shown in FIG. 3, the next step of determining a skill level of at least one cognitive ability may comprise creating a testing task 330. The testing task may be assigned to the user account may be operative to test one of the at least one cognitive ability and the response 350 received from the testing task 330 may determine the skill level of the at least one cognitive ability being tested 360.

In one embodiment, the first testing task 330 may be the same for all users. In another embodiment, the first testing task 330 may be determined by the user's post-illness impairment targeted profile 140 shown in FIG. 1B. For example, a user whose post-illness impairment targeted profile 140 comprise brain fog the first testing task may receive the first testing task operative to target the user's memory.

Returning to FIG. 3, in some embodiments, determining the skill level of the cognitive ability from the testing task 360 may regulate the creation of further testing tasks 330. Thus, the response to the training task 350 may determine whether there are more abilities to test 370. Following all cognitive abilities being tested 370, the skill level of at least one cognitive ability is determined 380 and the pre-calculated training 160 of FIG. 1B is assigned.

The final step shown in FIG. 1B comprises assigning the pre-calculated training 160. The pre-calculated training 160 may be determined by the skill level of at least one cognitive ability 150. It is contemplated that the pre-calculated 160 may be operative to rehabilitate at least one cognitive ability 150 that comprises a low skill level. In some embodiments, the one or more rehabilitation tasks 160 may comprise one or more rehabilitation tasks targeting at least one cognitive ability 150 that comprises an average or high skill level to reduce regression of at least one cognitive ability.

In another embodiment, a maximum tolerated dosage and a minimum effective dosage of effective training may be determined. The maximum tolerated dosage and the minimum effective dosage of effective training may be utilized in determining the pre-calculated training 160. For example, the pre-calculated training 160 may comprise one or more rehabilitation tasks above the minimum effective dosage. The pre-calculated training 160 may comprise one or more rehabilitation tasks below the maximum tolerated dosage. It is contemplated this may comprise one or more rehabilitation tasks below a maximum skill level or number of the one or more training tasks assigned. In one embodiment, a standard 3+3 rule, which will be recognized by one of ordinary skill in the art, may be utilized in determining the maximum tolerated dosage and minimum effective dosage. However, the maximum tolerated dosage and minimum effective dosage may be determined according to any manner that a person of ordinary skill in the art may recognize.

As shown in FIG. 1B, the steps of determining the skill level of at least one cognitive ability 150 and assigning one or more rehabilitation tasks 160 may be repeated. In some embodiments, these steps may be repeated to track progress of at least one cognitive ability. In a further embodiment, the step of determining the skill level of at least one cognitive ability 150 may be repeated in order to update the assigned one or more rehabilitation tasks 160.

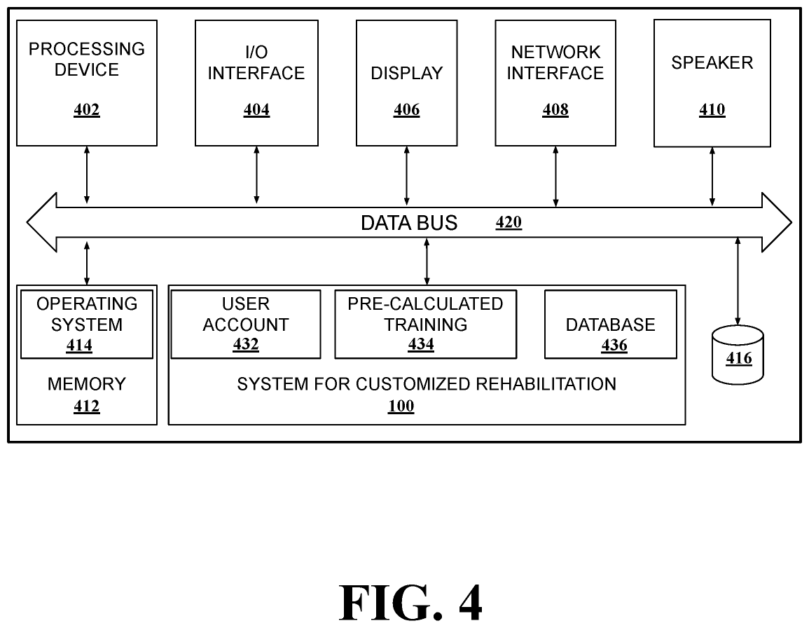

FIG. 4 illustrates an exemplary computing device 102 shown in FIG. 1A. As described earlier, computing device 102 may be a personal computer, tablet computer, laptop computer, or smartphone, but may also be embodied in any one of a wide variety of wired and/or wireless computing devices. As shown in FIG. 4 computing device 102 includes a processing device (processor) 402, input/output interfaces 404, a display 406, a network interface 408, a speaker 410, a memory 412, an operating system 414, and a mass storage 416 each communicated across a local data bus system 420. Additionally, computing device 102 incorporates a system for customizable cognitive rehabilitation 100, which is depicted as including a user account 432, a pre-calculated training 434, and a database 436, although the location of information 432, 434, and 436 could vary.

The processing device 402 may include any custom made or commercially available processor, a central processing unit (CPU) or an auxiliary processor among several processors associated with the mobile device 102, a semiconductor based microprocessor (in the form of a microchip), a macroprocessor, one or more application specific integrated circuits (ASICs), a plurality of suitably configured digital logic gates, and other electrical configurations comprising discrete elements both individually and in various combinations to coordinate the overall operation of the system.

The memory 412 can include any one of a combination of volatile memory elements (e.g., random-access memory (RAM, such as DRAM, and SRAM, etc.)) and nonvolatile memory elements. The memory typically comprises native operating system 414, one or more native applications, emulation systems, or emulated applications for any of a variety of operating systems and/or emulated hardware platforms, emulated operating systems, etc. For example, the applications may include application specific software which may comprise some or all the components of the computing device 102. In accordance with such embodiments, the components are stored in memory and executed by the processing device. Note that although depicted separately in FIG. 4, the system for customizable cognitive rehabilitation 100 may be resident in memory such as memory 412.

One of ordinary skill in the art will appreciate that the memory 412 can, and typically will, comprise other components which have been omitted for purposes of brevity. Note that in the context of this disclosure, a non-transitory computer-readable medium stores one or more programs for use by or in connection with an instruction execution system, apparatus, or device. With further reference to FIG. 4, network interface device 410 comprises various components used to transmit and/or receive data over a networked environment such as depicted in FIG. 1A. When such components are embodied as an application, the one or more components may be stored on a non-transitory computer-readable medium and executed by the processing device.

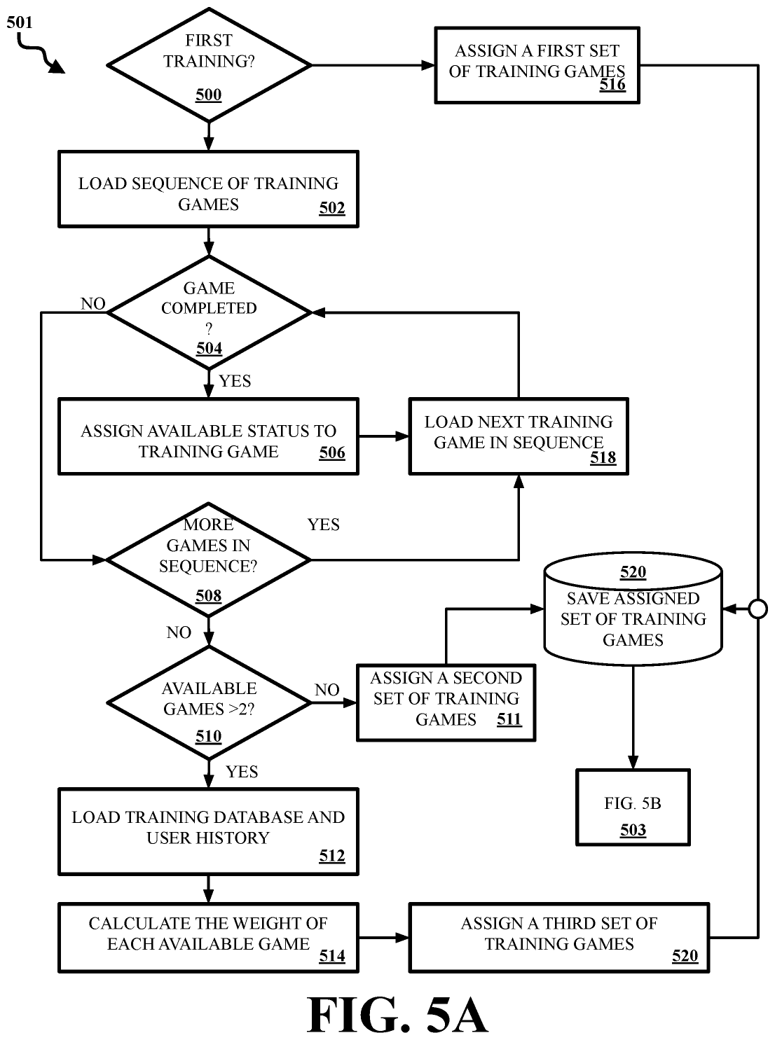

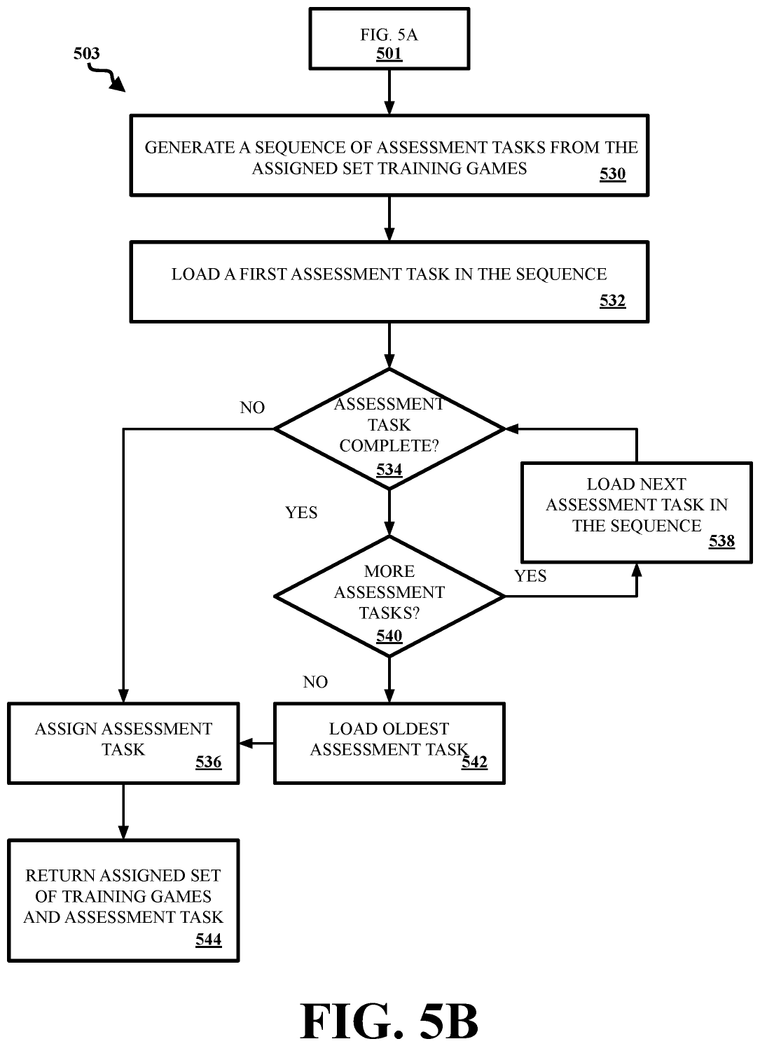

FIG. 5 illustrates an exemplary embodiment for determining at least one training game and at least one assessment task for the pre-calculated training 160 of FIG. 1B. It is contemplated that the pre-calculated training 160 may be operative to rehabilitate at least one cognitive ability. More particularly, the pre-calculated training 160 may be operative to rehabilitate the skill level of at least one cognitive ability 150 informed by the post-illness impairment targeted profile 140.

In some exemplary embodiments, as shown in FIG. 5, the precalculated training may comprise two training games and one assessment task. However, in some embodiments, not shown for the sake of brevity, the pre-calculated training 160 may comprise any number of training games and assessment tasks. In some such embodiments, the pre-calculated training 160 may comprise a ratio of at least one training game to at least one assessment task. For example, the ratio may be two training games to each assessment task. As yet a further example, the ratio may be 3:1, 3:2, 1:1, or 5:1, without limitation.

As shown in FIG. 5A, the pre-calculated training may comprise a first set of training games 516 if it is the user's first training. In some embodiments, the first set of training games 516 may be operative to rehabilitate at least one cognitive ability specific to the user. However, in another embodiment, the first set of training games 516 may be common for all users. Thus, the first set of training games 516 may be an established set of training games that are assigned without consideration of the post-illness impairment profile 140 or the skill level of at least one cognitive ability 150 determined in FIG. 1B. In some such embodiments, the first set of exemplary FIG. 5A training games 516 may be operative to train the user on how to operate the system. For example, the first set of training games 516 may comprise at least one training game and a displayed set of instructions to introduce different functions of the system and the training games.

The first set 516 of training games may be saved to a database 520. It is contemplated that by saving the first set 516 of training games to the database 520 the first set 516 of training games may be accessible on any computing device that accesses the user account.

As further shown in FIG. 5A, if the first training 500 has already been conducted, a sequence of training games 502 comprising a plurality of training games may be loaded. It is contemplated that a sequence of training games 502 may be ordered in a queue. In some embodiments, the sequence of training games 502 may be ordered depending on the complexity of the plurality of training games. However, in other embodiments, the sequence of training games 502 may be in random order. In further embodiments, the sequence of training games 502 may be ordered depending on the estimated time necessary for completion, compatibility with the computing device, length of time in the sequence, alphabetical order, or any other order that a person of ordinary skill may contemplate.

The sequence of training games 502 may be taken in a sequential order and the completion status 504 of the first game in the sequence may be determined. If the first training game has not been completed, then the training game may be assigned a status as an available training game 506. It is contemplated that completing the game may comprise partially completed games in addition to games that have not previously been accessed. In some embodiments, the training game may not be considered complete until all aspects of the training game have been finished. However, in other embodiments, the training game may be considered complete if 50% or more of the training game has been completed. Further, in another embodiment, a played training game may be considered not complete if a sufficient amount of time has passed. For example, the passage of a week, month, several months, or years may be sufficient to consider the played training game not completed. It is contemplated that the passage of time may warrant repetition by the user in order to retrain or maintain the skill level of the trained cognitive ability.

In some embodiments, not shown, the available training games may be determined based on the complexity of the training games and the user's skill level of at least one cognitive ability. In yet a further embodiment, the available training games may be determined by a variety of factors, such as the completion status, complexity, device compatibility, targeted cognitive ability, or any other factor. The variety of factors may be used alone or in combination to determine which of the plurality of training games are available.

In some embodiments, each of the plurality of training games in the sequence 502 of training games may be a unique training game. However, in another embodiment, any of the plurality of training games in the sequence 502 of training games may be the same training game. It is contemplated that in some such embodiments, the same training games may be identical, however, in other embodiments the same training games may comprise a different complexity and/or level than the training game.

Returning to FIG. 5A, if the training game has been completed 504 then the next step may be to determine if there are more training games in the sequence 508.

Regardless of the completion status 504 of the training game, if there are more training games in the sequence 508, the next training game 518 may be loaded and the step of determining the completion status 504 of the training game may be repeated. In some embodiments, the step of determining the completion status 504 of the training game may be repeated for each training game in the sequence. However, in another embodiment, the step of determining the completion status 504 may be repeated for less than all training games in the sequence.

Available training games 506 may be the training games eligible to be assigned. In some embodiments, as shown in FIG. 5A, the available training games may be determined by the completion status 504 of the training game. However, in another embodiment, the available training games may be the training games that target the at least one cognitive ability of the user that requires rehabilitation. In yet a further embodiment, the available training games 506 may be the training games that are unlocked, either through the completion of training games, by assignment by a third party, or according to any access level associated with the user account.

In some instances, the available training games may be less than or equal to a predetermined number of training games. As shown in FIG. 5A, the predetermined number of training games may be two (at 510). Thus, if the number of available games is less than or equal to two then all available training games may be assigned to a second set 511 of training games. However, the predetermined number of training games 510 may be any number of training games, such as one, three, four, five, or even more. The second set 511 of training games may be saved to the database 520.

In some instances, the available training games 506 may be greater than the predetermined number 510 of training games and less than all available training games 506 may be assigned to a third set 520 of training games. In one embodiment, a training database and user history may then be loaded 512. The training database 512a may comprise information relating to at least one training game. This information may comprise complexity, targeted cognitive abilities, estimated time to complete, and compatibility with the computing device relating to each of the at least one training game. In an exemplary embodiment, the training database may comprise a weight of the skill level of at least one tested cognitive ability by the available training game 506. In some embodiments, the training database may comprise information relating only to the available training games 506. However, in some embodiments, the training database may comprise the information relating to all of the at least one training game, regardless of the status of the training game.

In one embodiment, the user history may comprise any of the user's biographical information, post-illness cognitive impairment profile, and the user's skill level of at least one cognitive ability from. In some embodiments, the user history may further comprise the first assigned set of training games 516 and their results. In a further embodiment, the user history may comprise the results of previously assigned training games and assessment tasks.

The step of calculating a weight 514 of each available training game may utilize the training database and the user history. The weight 514 of each available training game may be assigned, selected, or calculated by any method known in the art. For example, the weight 514 of each available training game may be calculated by determining a relation weight between the skill level of at least one cognitive ability and one of the available games. The sum of the relation weight divided by the user's skill level of at least one cognitive ability may equal the weight of the game. However, a person of ordinary skill in the art may appreciate other methods of calculating the weight of the available training games. It is contemplated that the weight 514 of each available training game may be calculated individually. However, the weight 514 of each available training game may be calculated collectively and the collective value may influence each of the available training games.

In some embodiments, the weight 514 of each available training game may be calculated based on one of the at least one cognitive ability. However, in another embodiment, the weight 514 of each individual training game may be calculated based on multiple of the at least one cognitive ability. The multiple at least one cognitive ability may be determined by the post-illness impairment profile and the skill level of the at least one cognitive ability. Thus, the weight 514 of each available training game may reflect the available training games efficacy in rehabilitating the at least one cognitive ability

In some embodiments, the weight 514 of each available training may be calculated depending on the user's familiarity with the system. The number of training games previously played by the user may allow some of the available training games to be filtered out because of complexity. It is contemplated that calculating the weight 514 of each available training game depending on familiarity with the system may reduce frustration with the training games. Further, this may reduce the number of unfamiliar stimuli presented at once and/or unfamiliar inputs required in response to the training game. Thus, the accuracy of the training games may be increased because of a reduction in external factors that may impact the results.

In another embodiment wherein the available training games may comprise previously played training games, the weight given to training games that have not been completed may be greater than the weight of played training games. In other words, the system may be configured to prioritize training games that have not been completed over the played training games. In some embodiments, the previously played training games may only be assigned when all available training games have been played.

Returning to the embodiment illustrated in FIG. 5A, the available training games with the greatest calculated weight may be assigned to a third set of training games. The third set of training games may be equal to the predetermined number of training games. As shown in FIG. 5A, the predetermined number may, for example only, be two 510, and thus two training games having the greatest weight may be assigned.

In another embodiment, the assigned training games may have the lowest weight. As a clarifying example, when the weight is based on the complexity of the training game it may be advantageous to assign the training games with the lowest weight and thus the lowest complexity. In yet a further embodiment, the weight of the assigned training games may be within a desired range. For example, the weight may be within a range of the desired complexity of tasks to assign to the user and thus the training games assigned may comprise a greater efficacy in rehabilitating the at least one cognitive ability.

The third set of training games may be saved to the database 520.

With particular reference to FIG. 5B, a sequence of assessment tasks 530 may be generated from the assigned set of training games. In the instances where first set 516 of training games is saved to the database 520, the sequence of assessment tasks 530 may comprise at least one assessment task relating to the first set 516 of training games. In some embodiments, the sequence of assessment tasks 530 may comprise an established at least one assessment task and may thus be the same for all users.

In some such embodiments, each of the plurality of assessment tasks may be operative to assess the same cognitive ability as the training game. It is contemplated that though the plurality of assessment tasks may be operative to assess the same cognitive aspect may, nevertheless, be distinct assessment tasks. By providing distinct assessment tasks, it may prevent skill development on the assessment task that may incorrectly reflect the skill level of at least one cognitive ability.

The at least one assessment task may be operative to assess the skill level of at least one cognitive ability. In some embodiments, each of the at least one assessment tasks may be operative to assess the skill level of one cognitive ability. However, in other embodiments, each of the at least one assessment tasks may be operative to assess the skill level of multiple of the at least one cognitive ability.

In further embodiments, when the first set 516 of training games is assigned, the sequence 530 of assessment tasks may comprise only one assessment task. However, the sequence 530 of assessment tasks may comprise any number of assessment tasks generated from the first set of training games.

For brevity, the assigned set of training games is discussed as the second set of training games, however, a person of ordinary skill will appreciate that the second set of training games is used as an example, and the first set, third set, or any additional sets of training games are available. In instances where the second set of training games is assigned, the generated sequence 530 of assessment tasks may comprise at least one assessment task relating to the second set 511 of training games. In some embodiments, each of the at least one assessment tasks in the sequence 530 may be associated with only one training game in the second set 511 of training games. However, in another embodiment, at least one of the assessment tasks in the sequence 530 may be associated with any of the training games in the sequence 530. Thus, multiple of the at least one assessment tasks in the sequence 530 of assessment tasks may be related to multiple of the training games in the second set 511 of training games.

With continued reference to FIG. 5B, the first assessment task in the sequence of assessment tasks 530 may be loaded 532. The status of the first assessment task may then be determined 534. As shown in FIG. 5, the status may be whether the first assessment task has been completed. However, in other embodiments, not shown, the status may be another factor known in the art. For example, the status may be the availability of the assessment task to the user, the compatibility with the computing device, the length of time to complete the assessment task, or the cognitive ability assessed by the assessment task. In some embodiments, the status may be determined by one or more of the factors commonly known in the art.

Returning to FIG. 5B, when the first assessment task in the sequence is not completed, the first assessment task may be assigned 536. Continuing with the example wherein the second set of training games is assigned, the second set of training games and the assigned 536 assessment task may be returned 544. The returned 544 assigned training games and assessment task may comprise the pre-calculated training assigned to the user at 160 in FIG. 1B.

However, returning to FIG. 5B, if the first assessment task in the sequence is completed, the next step may comprise determining if there are additional assessment tasks in the sequence of assessment tasks 540. If there are additional assessment tasks in the sequence, the next assessment task in the sequence of assessment tasks may be loaded 538. The next assessment task may repeat the step of determining the status 534 of the assessment task 538. If the next assessment task has not been completed, the next assessment task may be assigned 538. It is contemplated that the steps of determining if there are additional assessment tasks in the sequence 540 and loading the next assessment task 538 in the sequence may be repeated until the loaded assessment task has not been completed and may be assigned 536. The assigned assessment task, and, continuing with the previous examples, the second set of training tasks may be returned 544. The returned 544 assigned set of training game and assessment tasks may comprise the pre-calculated training assigned at 160 in FIG. 1B.

Turning once again to FIG. 5B, if all the assessment tasks in the sequence 530 of assessment tasks have been completed 534, then the completion date of each of the assessment tasks in the sequence may be determined. The assessment task in the sequence of assessment tasks having the oldest date of completion may then be loaded 542 and assigned 536. However, in some embodiments, not shown, the assigned assessment task may be determined based on a variety of factors, such as the relationship to the assigned training games, the results from the assessment task when completed previously, or a calculated weight of the assessment tasks. A person of ordinary skill in the art will appreciate that the aforementioned factors are provided for example only and any factor may be utilized.

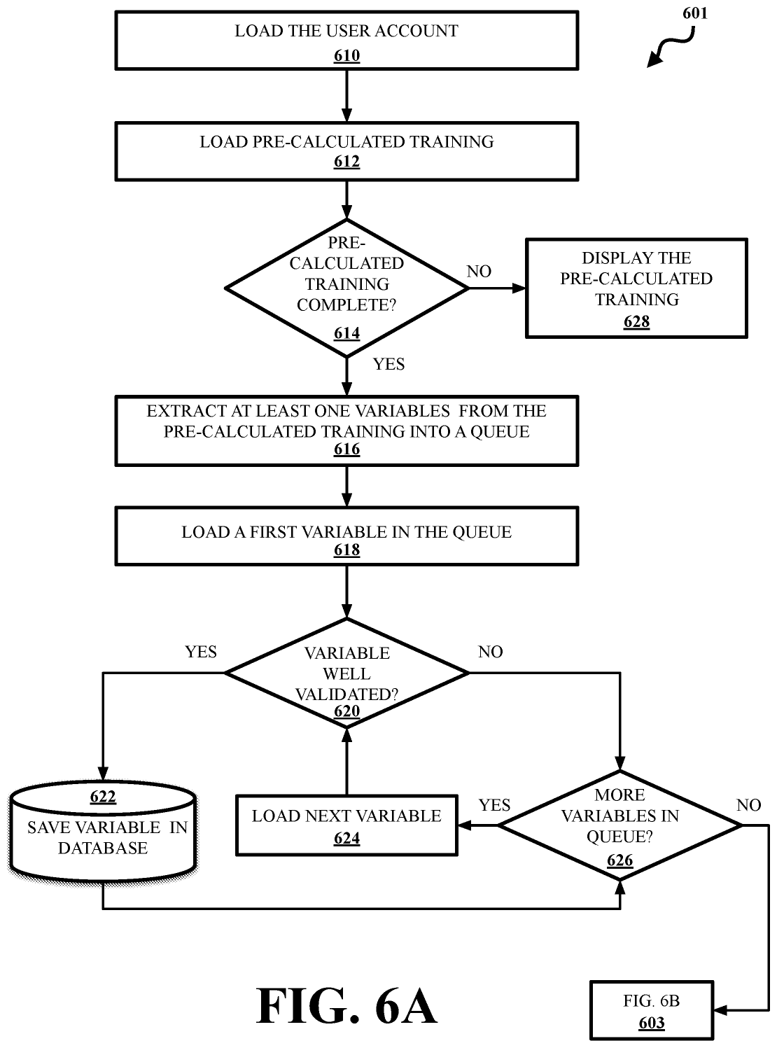

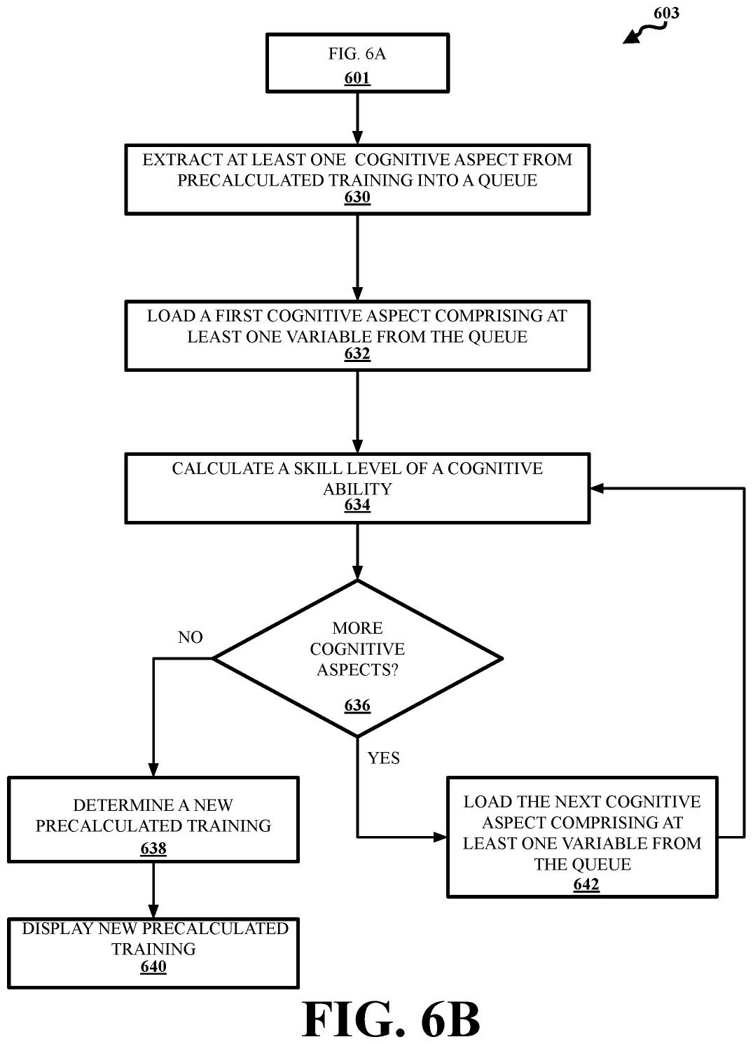

FIGS. 6A and B illustrate one aspect of the method for customizable cognitive rehabilitation as steps for calculating the skill level of at least one cognitive ability. As shown in FIG. 6A, the first step in calculating the skill level of at least one cognitive ability may be loading the user account 610. It is contemplated that the loaded user account 610 may be the same user account created at 115 in FIG. 1B. As shown in FIG. 1B, the pre-calculated training is assigned 160 to the user and may be the loaded pre-calculated training 612 in FIG. 6A. Thus, in some embodiments, the loaded pre-calculated training may comprise the returned 544 assigned set of training games and assessment tasks from FIG. 5B.

Returning to FIG. 6A, the system may be operative to determine a completion status 614 of the loaded pre-calculated training. In one exemplary embodiment, the loaded pre-calculated training may be complete when all aspects of such pre-calculated training have received a response from the user. For example, when the pre-calculated training comprises two training games and one assessment task, the pre-calculated training may be complete when a response has been received for each of the two training games and one assessment task. However, in another embodiment, the pre-calculated training may be complete if the at least one assessment task is complete. In yet a further embodiment, the pre-calculated training may be complete if the at least one training game is complete. It is contemplated that the pre-calculated training may be complete in any number of circumstances and the aforementioned are provided as non-limiting examples.

If the loaded pre-calculated training has not been completed 614, the pre-calculated training may be displayed 628 on any of the computing devices 102 and 104 as illustrated in FIG. 1A. In some embodiments, the display may include a prompt instructing the user to complete the pre-calculated training as in block 614 of FIG. 6A. In a further embodiment, the display may be operative to receive a response from the user requesting a new pre-calculated training. This is contemplated to be beneficial as a prompt to the system to re-calculate the pre-calculated training to be customized to the user's needs at the time. For example, when the pre-calculated training comprises an audio component, such as the playing of a sound, the system may be operative to assign a new training, in response to a user request, that does not comprise sound where emitting or generating sound is undesirable, impossible, or even impractical for any reason.

Continuing with FIG. 6A, when the status of the pre-calculated training 614 is complete, at least one variable may be extracted from the pre-calculated training and placed in a queue 616. In some embodiments, the queue may comprise all variables present in the pre-calculated training. However, the queue may, in other embodiments, comprise less than all the variable present in the pre-calculated training. For example, the queue may only comprise variables relating to the at least one cognitive ability targeted by the pre-calculated training.

The at least one variable in the queue may be arranged in any order, such as a random, ordered by a time when variable was received, grouped with like variables, or grouped with other variables received from the same game and/or task.

A first variable from the queue may then be loaded 618. The system will consider whether the first variable is well validated 620. The variable may be considered well validated when the variable comprises a value within an established range for the variable. In some embodiments, the variable is considered well validated 620 when it is within the established range with variables extracted from previous pre-calculated trainings. In another embodiment, the established range may be calculated from values of the variables extracted for all users. In yet a further embodiment, the established range may be within a median value of the variable for users having the same or similar illness. Thus, the first variable may be considered to not be well validated if it is outside the established range.

In some exemplary embodiments, the established range may be a 95% confidence value. However, in other embodiments, the established range may be, without limitation, a confidence value between 70% to 99%, inclusive. In some embodiments, the established range may be a 70%, 75%, 80%, 85% 90%, 95%, 98%, or 99% or higher confidence value. A person of ordinary skill will recognize that the provided confidence values are provided for example only, and any value may be utilized. In one exemplary embodiment, the validation may utilize Cronbach's Alpha, however, a person of ordinary skill will appreciate that any known statistical method may be utilized.

If the first variable in the queue is well validated it may be saved in the database 622. The next step may then be to determine whether there are more variables in the queue 626. The step of determining whether there are more variables in the queue 626 may additionally be the next step when the first variable is not well validated at 620.

If there are more variables in the queue 616, the next variable 624 may be loaded, and the step of determining if the variable is well validated 620 may be repeated in turn for each variable in the queue. Thus, each variable in the queue that is well validated at 620 may be saved in the database 622.

It is contemplated that, in some embodiments, the variable may be well validated when the next variable in the queue is within the established range to the first variable. It is further contemplated that the variable may be well validated when the at least one variable in the queue is within the established range to any of the other at least one variable in the queue.

In one embodiment, the confidence interval of the established range may evolve as the method is performed by the system for customizable cognitive rehabilitation. Thus, it is contemplated that the confidence interval may have a lower value during a first iteration of the method, than during later iterations of the method. Allowing an evolving established range may allow the system to become more accurate as more data is collected. As a clarifying example, when the pre-calculated training comprises the exemplary first set of training games referenced in FIG. 5A, the variable may be well validated if a calculated confidence value is greater than 70%. Continuing with the example, when the pre-calculated training comprises the second set or the third set of training games, the variable may be well validated if the calculated confidence value is greater than 95%. In some embodiments, the calculated confidence value may increase as more variables are extracted. In another embodiment, the confidence value of the established range may be the same for each iteration of the method.

Returning to FIG. 6A, as previously noted, if the variable is well validated, the variable may be saved in the database 622. Thus, it is contemplated that saving the variable in the database 622 may update values utilized in calculating the confidence value. That is, as more well validated variables are extracted, the system become get more precise.

After each variable in the queue has been loaded 618, 624 and the validation of each variable is determined, the system may, as in FIG. 6B, extract at least one cognitive aspect of at least one cognitive ability targeted by the pre-calculated training into a queue of cognitive aspects 630. The cognitive aspects are contemplated to be discrete components of the response received from the pre-calculated training. For example, the cognitive aspects of the received response may comprise attention, perception and recognition, memory, learning, reading, speaking, listening, problem-solving, planning, reasoning, and decision making. However, a person of ordinary skill will appreciate the aforementioned cognitive aspects are provided as non-limiting examples and any cognitive aspects may be utilized.

The at least one variable comprised by the cognitive aspect may be the at least one variable extracted in FIG. 6A. In some embodiments, each exemplary FIG. 6B cognitive aspect in the queue may comprise one variable. However, in other embodiments, each cognitive aspect in the queue may comprise multiple of the at least one variable.

In some embodiments, the pre-calculated training may target only one cognitive ability. However, in another embodiment, the pre-calculated training may target multiple of the at least one cognitive ability. In either embodiment, the pre-calculated training may comprise at least one cognitive aspect relating to the cognitive ability. For example, when the pre-calculated training is targeting short-term memory, the at least one cognitive aspect may be attention, reading, recognition, or listening, among any other cognitive aspects that may be contemplated.

In some embodiments, the queue of cognitive aspects may be ordered by the cognitive ability being tested. In another embodiment, the queue 630 of cognitive aspects may be ordered by the response received. In yet a further embodiment, the queue of cognitive aspects may have the at least one cognitive aspect in any random order. It is contemplated that the aforementioned orders are provided as non-limiting examples and any order may be utilized.

Following the extraction of at least one cognitive aspect from precalculated training into a queue of cognitive aspects 630 (in short, generation of the queue), the first cognitive aspect comprising at least one variable may be loaded 632. The at least one variable of the first cognitive aspect may be utilized to calculate the skill level of the at least one cognitive ability 634 targeted by the pre-calculated training. The skill level of the at least one cognitive ability may be utilized to track cognitive rehabilitation of the user. It is contemplated that calculating a skill level of the cognitive ability 634 may provide easily comprehensible results to the user. Indeed, doing so may promote a user's or even third party's feeling of progress and accomplishment that may serve to increase compliancy with the cognitive rehabilitation program.

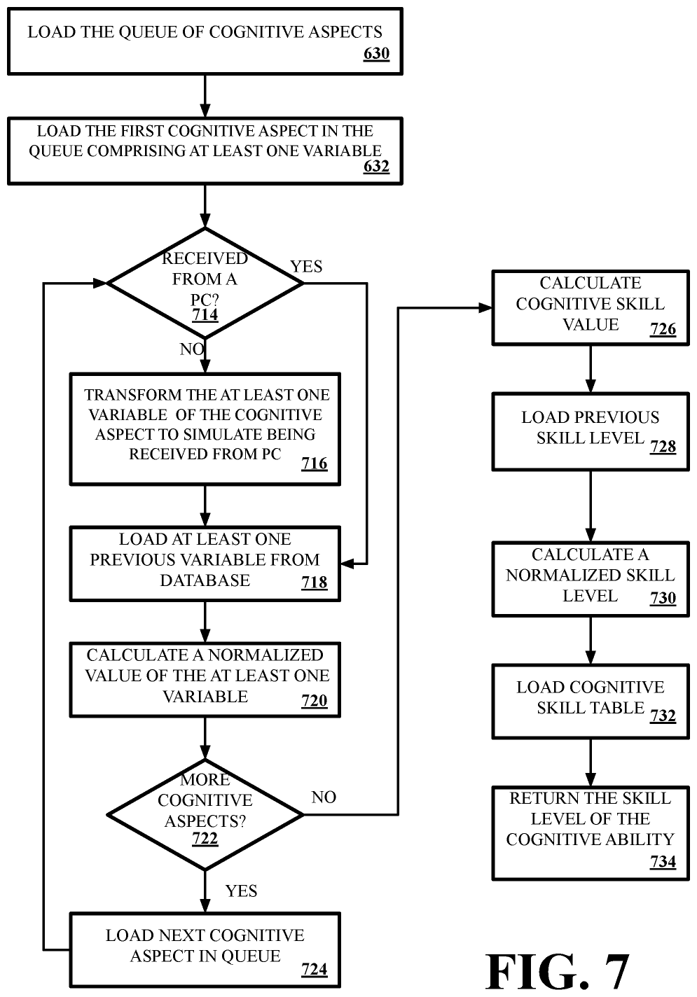

FIG. 7 provides one exemplary embodiment of calculating the skill level of the at least one cognitive ability 634 from FIG. 6B. As shown in FIG. 7, the first cognitive aspect in the queue comprising at least one variable may be loaded 632. It may then be determined whether the at least one variable of the cognitive aspect was received from a personal computer 714. By determining whether the at least one variable of the cognitive aspect was received from the personal computer, the system may be operative to transform the at least one variable to simulate being collected on the personal computer 716. Thus, the at least one variable collected on a different computing device, such as a smartphone, may be uniformly compared to the at least one variable collected on the personal computer. The personal computer may be utilized in the exemplary embodiment shown in FIG. 7, however, any computing device may be utilized. For example, the variable may be transformed to reflect being collected on a smartphone, tablet computer, laptop computer, or any other computing device in the art. In yet a further embodiment, not shown, all of the at least one variable may be transformed to non-device specific variables.

Returning to FIG. 7, the step of transforming the at least one variable 716 may occur when the at least one variable is received from a device other than the personal computer 714. If the at least one variable is received from the personal computer 714, the step of transforming 716 may not be necessary to allow for the uniform comparison of the at least one variable.

After the at least one variable is in a condition to allow for uniform comparison, at least one previous variable may be loaded from a database 718. The at least one previous variable may, in one embodiment, be at least one variable extracted from the user's previous pre-calculated training. In another embodiment, the at least one previous variable may be at least one variable from one or more other users' pre-calculated trainings. In some such embodiments, the at least one previous variable may at least one variable from other users' pre-calculated training having the same or similar illness. It is contemplated that the at least one previous variable may comprise a combination of at least one variable from the user and other users.

It is contemplated that the at least one previous variable 718 may be the same as the at least one variable from the first cognitive aspect referenced at 632. In some embodiments, the at least one variable may be from the same cognitive aspect as the at least one previous variable. In a further embodiment, the at least one variable may be from the same cognitive aspect of the same cognitive ability as the at least one previous variable. In yet a further embodiment, the at least one variable may be from the same assessment task as the at least one previous variable.

After the at least one previous variable is loaded 718 a normalized value may be determined for the at least one variable 720. The normalized value may be calculated by any known method of data normalization known in the art. For example, the at least one variable may be normalized by taking a value of the variable, either the transformed or original value, and subtracting an average value of the at least one previous variable and dividing by a variance value. A returned value from the previous calculation may then, in some embodiments, be weighted to calculate the normalized value 720. A person of ordinary skill will appreciate that this is only one of innumerable methods of calculating normalized values and all known methods may be utilized.

Following the step of calculating the normalized value of the first variable 720, the next step may be to determine if there are more cognitive aspects in the queue of cognitive aspects 630, as in FIG. 6B. If there are more cognitive aspects in the queue 630 of cognitive aspects, the next cognitive aspect in the queue of cognitive aspects may be loaded 642. The steps of determining if the cognitive aspect is received on the personal computer 714, transforming the at least one variable from the cognitive aspect if received on a computing device that is not the personal computer 716, loading at least one previous variable from the database 718, and calculating the normalized value of the variable 720, may be repeated for each of the at least one cognitive aspect in the queue of cognitive aspects 630.

Once the normalized value has been calculated 720 for each cognitive aspect in the queue of cognitive aspects 630, a skill value of at least one cognitive ability may be calculated 726. In some embodiments, the skill value of at least one cognitive ability may be the sum of the normalized value from the queue of cognitive aspects. However, a person of ordinary skill will appreciate other forms of calculating the at skill value of at least one cognitive ability.

A previously calculated skill value of at least one cognitive ability may be loaded from the database 728. In one embodiment, the previously calculated skill value of at least one cognitive ability may be specific to the user. However, in another embodiment, the previously calculated skill value of at least one cognitive ability may be an arbitrary value. It is contemplated that in some instances, such as when it is the user's first training, the previously calculated skill value may be a calculated skill value of users having the same or similar illness.

A normalized skill value may be calculated from the skill value 730 of at least one cognitive ability and the previously calculated skill value. Any method of normalizing values may be utilized, such as subtracting the previously calculated skill value from the skill value of at least one cognitive ability and dividing by a variance value.

A cognitive skill table may be loaded 732 and may comprise percentages relating to the normalized skill value. In some embodiments, the cognitive skill table may be specific to each discrete cognitive ability. However, in another embodiment, the cognitive skill table may be universal for all cognitive abilities. Further, in some embodiments, the cognitive skill table may be based on the user. However, in other embodiments, the cognitive skill table may be universal for all users.

The skill level of the cognitive ability may be a percentage relating to the normalized skill value on the cognitive skill table. In some embodiments, the percentage value of 100% may represent the reported pre-illness cognitive levels. In another embodiment, the percentage value of 100% may represent an expected skill level of at least one cognitive ability once rehabilitated.

The skill level of the cognitive ability may be returned 734. In some embodiments, the returned skill level of the cognitive ability may be displayed on the computing device. In another embodiment, the returned skill level of the cognitive ability may be updated in the user account.

Returning to FIG. 6B, following the step of calculating the value of the first skill 634, the queue 630 of cognitive aspects may be evaluated to determine whether there are additional cognitive aspects. If there are additional cognitive aspects in the queue of cognitive aspects, the next variable may be loaded 642. The value of the next cognitive aspect may then be calculated 634, as shown in the method of FIG. 7. It is contemplated that each of the cognitive aspects in the queue of cognitive aspects may be calculated in this manner. Once all the cognitive aspects in the queue of cognitive aspects have been calculated 634, a new pre-calculated training may be determined 638. In an exemplary embodiment, the new pre-calculated training may utilize the method illustrated in FIG. 5. The new pre-calculated training may then be displayed 640 on the at least one computing device 102 and 104 illustrated in FIG. 1A.

### CONCLUSIONS, RAMIFICATIONS, AND SCOPE

While certain embodiments of the invention have been illustrated and described, various modifications are contemplated and can be made without departing from the spirit and scope of the invention. For example, the illness may be a disease such as dementia or Alzheimer's, and thus the system and method may be utilized to slow the progression of the disease. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the cognitive rehabilitation method and system for illness-related cognitive impairment with which that terminology is associated. In general, the terms used in the following claims should not be constructed to limit the cognitive rehabilitation method and system for illness-related cognitive impairment to the specific embodiments disclosed in the specification unless the above description section explicitly defines such terms. Accordingly, the actual scope encompasses not only the disclosed embodiments, but also all equivalent ways of practicing or implementing the disclosed system, method and apparatus. The above description of embodiments of the cognitive rehabilitation method and system for illness-related cognitive impairment is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the cognitive rehabilitation method and system for illness-related cognitive impairment.

What is claimed is:

1. A method for customizable cognitive rehabilitation comprising:
by an electronic device,
receiving biographical information;
creating a user account;
conducting a post-illness cognitive impairment battery comprising receiving information on the user's sociodemographic factors, illness, pre-illness cognitive levels, and post-illness cognitive levels;
creating from the post-illness cognitive impairment battery a post-illness cognitive impairment targeted profile;
determining a skill level of at least one cognitive ability comprising:
presenting at least one stimulus;
receiving at least one motion input;
analyzing at least one cognitive aspect;
assigning a precalculated training operative to rehabilitate at least one cognitive ability.

2. The method of claim 1, wherein analyzing the at least one cognitive aspect comprises:
testing a preliminary cognitive level;
receiving at least one result; and
determining at least one discrete skill level of the cognitive ability.

3. The method of claim 1, wherein analyzing the at least one cognitive aspect comprises:
creating a testing task;
assigning the testing task to the user account;
receiving a response to the testing tasks; and
determining the skill level of the cognitive ability associated with the testing task.

4. The method of claim 3, wherein the post-illness cognitive impairment profile comprises at least one targeted cognitive ability and the step of analyzing the at least one cognitive aspect is repeated for each cognitive ability in the post-illness cognitive impairment profile.

5. The method of claim 1, wherein the precalculated training comprises at least one training game and at least one assessment task.

6. The method of claim 5, wherein the precalculated training comprises two training games and one assessment task.

7. The method of claim 1, further comprising updating the skill level of at least one cognitive ability and assigning a new precalculated training comprising:
receiving the results of the precalculated training;
calculating a new skill level of the at least one cognitive ability;
loading a sequence of training games;
assigning at least one training game from the sequence of training games to the new precalculated training;
loading a sequence of assessment tasks; and
assigning at least one assessment task associated with the assigned at least one training game to the new precalculated training.

8. The method of claim 7, wherein the assigned at least one training game is selected from the sequence of training games by:
loading each training game sequentially;
determining whether the loaded train game is an available training game;
calculating the weight of each available training game; and
assigning the available training games with the greatest weight.

9. The method of claim 1, further comprising:
receiving a response to the precalculated training; and
transforming the response to simulate a response collected on a personal computer.

10. The method of claim 1, further comprising:
loading a cognitive skill percentile table from a database;
calculating a skill value from a response to the precalculated training;
locating the skill value on the skill percentile table; and
returning from the skill percentile table a cognitive skill level.

11. A system for digital assessment and rehabilitation of cognitive abilities comprising:
a first computing device operative to:
receive a user's information;
create a user account associated with the user's information;
receive a post-illness cognitive impairment battery;
responsive to receiving the post-illness cognitive impairment battery, generate a post-illness impairment targeted profile;
perform a method to determine a skill level of at least one cognitive ability comprising:
presenting at least one stimulus;
receiving at least one motion input in response to the at least one stimulus; and
analyzing at least one cognitive aspect associated with the at least one motion input;
assign a precalculated training operative to rehabilitate at least one cognitive ability;
present, on the first computing device, the precalculated training; and
transmit to a database the user account, the skill level of at least one cognitive ability, and the precalculated training.

12. The system of claim 11, further comprising a second computing device operative to:
access the user account;
present the precalculated training;
receive a response to the precalculated training;
responsive to receiving the response, transform the response to simulate responses collected on the first computing devices; and
transmit the transformed response to the database.

13. The system of claim 11, wherein analyzing the at least one cognitive aspect comprises:
testing a preliminary cognitive level;
receiving at least one result; and
determining at least one discrete skill level of the cognitive ability.

14. The system of claim 11, wherein analyzing the at least one cognitive aspect comprises:
creating a testing task;
assigning the testing task to the user account;
receiving a response to the testing task; and
determining the skill level of the cognitive ability associated with the testing task.

15. The system of claim 14, wherein the post-illness cognitive impairment profile comprises at least one targeted cognitive ability and the step of analyzing the at least one cognitive aspect is repeated for each cognitive ability in the post-illness cognitive impairment profile.

16. The system of claim 11, wherein the first computing device is further operative to:
access the user account;
present the precalculated training;
receive a response to the precalculated training;
repeat the method of determining the skill level of the at least one cognitive ability;
assign a new precalculated training;
transmit the skill level of the at least one cognitive ability and new precalculated training to the database.

17. The system of claim 11, wherein the first computing device is further operative to:
receive a response to the precalculated training;
load a skill percentile table from the database;
calculate a skill value of at least one cognitive ability from a cognitive aspect of the response to the precalculated training; and
return the skill level of the at least one cognitive ability from the percentiles table.

18. The system of claim 11, wherein the precalculated training comprises at least one training game and at least one assessment task.

19. The system of claim 11, wherein the precalculated training comprises two training games and one assessment task.

20. A non-transitory, tangible computer-readable medium having stored thereon computer-executable instructions, which, when executed by a computer processor, enable performance of the method comprising:
receiving, at a first computing device, user information;
responsive to receiving user information, create a user account;
conducting a post-COVID-19 cognitive impairment battery comprising receiving information on the user's sociodemographic factors, medical history, pre-COVID-19 cognitive levels, and post-COVID-19 cognitive levels;
creating a post-COVID-19 cognitive impairment profile from the post-COVID-19 cognitive impairment battery;
determining a skill level of at least one cognitive ability comprising the following steps:
a.) presenting, at the firsts computing device, at least one stimulus;
b.) receiving, at the first computing device, at least one motion input; and
c.) analyzing at least one cognitive aspect associated with the at least one motion input; and
assigning a precalculated training comprising two training tasks and one assessment task.
